(19) United States
(12) Patent Application Publication
(10) Pub. No.: US 2026/0069481 A1
(43) Pub. Date: Mar. 12, 2026

(54) DEVICE INCLUDING A PRESSURE CHAMBER AND A PUMP

(71) Applicant: L&R Kältetechnik GmbH & Co. KG, Sundern (DE)

(72) Inventor: Lothar Abels, Nörvenich (DE)

(21) Appl. No.: 19/314,865

(22) Filed: Aug. 29, 2025

Related U.S. Application Data
(63) Continuation of application No. PCT/EP2024/055137, filed on Feb. 28, 2024.

(30) Foreign Application Priority Data
Mar. 1, 2023 (DE) 102023105083.4
May 8, 2023 (DE) 102023111990.7

(51) Int. Cl. A61G 10/02; G05B 19/042; G16H 40/63

(52) U.S. Cl. CPC A61G 10/023; G05B 19/042; G16H 40/63; A61G 2200/327; G05B 2219/23399

(57) ABSTRACT

A device includes: a pressure chamber including a fastener movable between an open state and a closed state; a pump to pump gas into the pressure chamber to increase pressure within the pressure chamber and to pump gas out of the pressure chamber to reduce pressure within the pressure chamber; and a lying surface including a first part arrangeable in the pressure chamber and a second part arranged outside the pressure chamber, wherein the fastener, in the closed state, is configured to close the pressure chamber around a human body arranged on the lying surface in an airtight manner in relation to an environment.

### CROSS REFERENCE TO RELATED APPLICATIONS

This application is a continuation of International Application No. PCT/EP2024/055137, filed on Sep. 6, 2024, which claims priority under 35 U.S.C. § 119(a)-(d) to German patent application No. 10 2023 105 083.4, filed on Mar. 1, 2023, and German patent application No. 10 2023 111 990.7, filed May 8, 2023, the entire contents of which are hereby incorporated by reference.

### TECHNICAL FIELD

The present disclosure relates to a device with a pressure chamber and a pump, in particular for performing an alternating pressure massage on a user.

### BACKGROUND

DE 199 12 611 A1 discloses a vacuum chamber that can be used to treat a human body. The vacuum chamber stands on the floor and includes an entrance opening at the top. A hose made of substantially flexible material is attached to the entrance opening. The tube can be pulled over the torso of the body like a skirt from the clothing industry and then twisted. The twisting of the hose causes the fabric to wrap tightly around the torso and especially the abdomen. The twisted hose seals the vacuum chamber airtight. During operation, the pressure in the vacuum chamber can be lowered to treat the human body.

Herein, it is disadvantageous that this type of seal works only when the vacuum chamber is upright and the entrance opening is facing upwards. Therein, the vacuum chamber must be entered from above. For one thing, entering the upright vacuum chamber is very uncomfortable, as the user has to raise their legs very high to enter the vacuum chamber. Especially within the narrow dimensions of the vacuum chamber, it is not possible to provide an entrance aid in the form of a stool or the like that the user can lean on and which can be easily removed between the chamber wall and the user after entry. Further, a seal on the user's torso or abdomen within a version of the standing vacuum chamber can be realized only with certain size dimensions of the user, i.e., a very large user and a very small user cannot use the same vacuum chamber.

### SUMMARY

In contrast, the disclosed device is based on the objective of being usable for treating the human body regardless of the user's height.

A device according to the disclosure comprises a pressure chamber and a pump. The pressure chamber has a fastener that can be switched from an open to a closed state and vice versa. When closed, the fastener is designed to seal the pressure chamber around a human body positioned on the lying surface in an airtight manner with respect to the environment. The pump is designed to pump a gas into the pressure chamber and thus increase the pressure inside the pressure chamber and to pump it out of the pressure chamber and thus reduce the pressure inside the pressure chamber. Further, a reclining surface is comprised, wherein a first part of the lying surface is arranged in the pressure chamber and wherein a second part of the lying surface is arranged outside the pressure chamber.

The device according to the disclosure has many advantages. A significant advantage of the device is that a user can lie down on the lying surface so that the legs and lower body can be comfortably inserted into the pressure chamber, which for this purpose extends substantially along a horizontal direction. Furthermore, the dimensions of the pressure chamber can be adapted to the maximum body dimensions of the user. The user can then position themselves on the lying surface, regardless of their body dimensions, such that the fastener can seal the torso and especially the abdomen. Advantageously, this provides a device that enables cosmetic application or therapy independent of the user's body dimensions.

The device enables improving the physical appearance, especially the appearance of the skin. The pump achieves an (alternating pressure) massage by applying negative pressure and/or positive pressure to the lower body. Beyond that, through the application, a stimulation of the lymph flow as well as a positive influence on the connective tissue can be achieved.

The pressure chamber has a fastener which, when closed, is designed to seal the pressure chamber around a human body positioned on the lying surface in an airtight manner with respect to the environment. In this context, the term “airtight” is in particular understood to mean that it is possible to achieve and maintain a negative or positive pressure of more than 30 mbar within the pressure chamber by means of the pump. Advantageously, a negative pressure of up to −130 mbar is possible. Therein, the pump is designed to pump a gas into or out of the pressure chamber. In the context of this description, a gas is also understood to mean, in particular, a gas mixture such as (ambient) air. The gas is preferably air.

Preferably, the pump comprises at least one side channel compressor. Advantageously, the side channel compressor can achieve the required pressure level within the pressure chamber. Beyond that, other pumps are also possible.

Preferably, the pressure chamber comprises LED strips on the outside. Advantageously, the use of at least one LED strip enables providing visual feedback to the user and operating personnel about the operating status of the pressure chamber. For this purpose, the LED strip can, for example, turn on and off (pulsate) periodically during the massage application to indicate the massage mode, while the LED strip, for example, lights up continuously at the end of the application. Color codes can also be used to indicate the current operating status (e.g., red: occupied or in operation, green: unoccupied and usable).

The pressure vessel or pressure chamber is preferably made of carbon and is therefore particularly light and robust.

Preferably, the pressure chamber designed as a pressure vessel can be moved from a first state to a second state and vice versa, in particular along a substantially horizontal direction. The pressure vessel or pressure chamber can be moved manually or electrically for convenient positioning of the user and is therefore particularly suitable for people with disabilities. In the first state of the pressure chamber, more than three-quarters, preferably the entirety, of the length of the first part of the lying surface is arranged inside the pressure chamber. In particular, in the second state of the pressure chamber, less than half of the length of the first part of the lying surface is located outside the pressure chamber (also known as the pulled-out state). Advantageously, a method enables particularly convenient access and use of the device, as a user can position themselves comfortably on the lying surface and then be retracted into the pressure chamber with the lying surface. Through that, the device can in particular also be comfortably used by people with limited mobility.

Particularly preferably, the pressure vessel can be moved relative to a base frame via a rail structure. This can be done manually or electrically via an actuator. The two lying surfaces are attached to the base frame, in particular fixedly. The advantage of this is that the user's orientation does not change in relation to the base frame when entering and exiting the device. This means that the user's center of gravity (when used correctly) is always inside the base frame. Through this, the risk of the device tipping over when entering and exiting the pressure chamber and, in particular, when lying down on the lying surface, is minimized or, in particular, theoretically even completely prevented.

In particular, an inclination of the second part of the lying surface relative to the first part can be adjusted. Advantageously, the second part of the lying surface can be angled in relation to the first part so that the user can use the second part of the lying surface as a backrest. Thus, the user can first sit down, as with a seat, at first with the second reclining surface angled and the second reclining surface can subsequently be moved into a reclining position with the user's upper body. Further, a desired inclination of the second lying surface can also be set during treatment if this is felt to be comfortable.

Preferably, the first and second lying surfaces are height-adjustable, preferably jointly or individually. Advantageously, through that, the height of the lying surface can be optimized for the user so that they can easily climb onto the lying surface. Further, it is also possible to adjust the height of the lying surfaces such that the sealing effect is optimized by the fastener by aligning the height of a body axis in relation to the fastener.

Preferably, the first and second lying surfaces are touching each other. Preferably, the first and second lying surfaces are firmly connected to each other. Preferably, the first and second lying surfaces are in permanent contact. Advantageously, a continuous lying surface is present. Advantageously, in particular continuous support in the lower back area can be achieved through that. Besides that, in particular a uniform height level at the transition between the first and second lying surface can be ensured.

Expediently, the fastener comprises a fastening ring. In particular, the fastener can be moved from the open state to the closed state by rotating the fastening ring about a central axis or axis of symmetry in any one of two directions of rotation. Advantageously, particularly comfortable operation of the fastener is enabled through that. Preferably, the fastening ring is rotatably mounted on the pressure chamber. In particular, the fastening ring is mounted through a bearing on the pressure chamber. Preferably, the bearing comprises a large number of ball bearings, so that the fastening ring can rotate particularly smoothly. In particular, the fastening ring is made of aluminum and is therefore particularly light. Preferably, the fastener comprises a flexible fabric mechanism made of an airtight or gas-tight material, which is in particular tubular in shape. In particular, the flexible hose wraps around the user's torso by twisting or twirling and forms an “airtight” seal against it.

Advantageously, the fabric mechanism has a support length of a contact surface between 2 and 20 cm, preferably between 4 and 15 cm and particularly preferably between 6 cm and 10 cm. In an advantageous embodiment, the fabric mechanism has a support length of approximately 8 cm (+−20%). In particular, the support length can be more than 25 cm, 30 cm, or even 40 cm. Here, the contact surface is the surface available for contact with the user's torso. Advantageously, the long contact length in relation to systems known from prior art, with a contact length of 0.5 cm to 1 cm, enables particularly good sealing of the user's torso against the environment. Beyond that, the pressure exerted on the torso is low due to the large contact surface, so that the fabric mechanism is not perceived as “cutting”. This makes it possible to achieve negative and positive pressures of up to ±130 mbar in the pressure chamber during operation of the device.

Advantageously, the lying surface is at least partially curved in a direction transverse to the direction of travel of the lying surface. Advantageously, through that it is achieved that the fastener with the airtight fabric mechanism can seal itself even better, in particular on the sides of the lying surface.

In particular, an inclination of the lying surface can be adjusted so that, for example, the feet of a user are raised in relation to the torso. Advantageously, such a negative angle can be 5° or 10° or 20° or more. Advantageously, during operation of the device, a lymphatic reflux, particularly from the user's legs, is preloaded. Advantageously, the effectiveness of the treatment is further improved through that.

Preferably, the fastening ring has a plurality of recesses along its circumference, wherein the fastener comprises a fixing bolt, and wherein the fixing bolt is designed to prevent rotation of the fastening ring when it is arranged in one of the recesses, also referred to as a quick latch. In particular, the recesses are arranged on an axial front side of the locking ring.

In particular, a recess has a round, in particular circular cross-section. Advantageously, the fixing bolt has a tapered section, in particular the fixing bolt is designed as a truncated cone, which preferably tapers conically along a central axis. The fixing bolt can be conveniently moved parallel to a central axis of the fastening ring and inserted into the recess. In particular, a displacement leads to the fastening ring being arrested or fixed in place or the fastening ring being released.

Preferably, the fastener comprises a rocker mechanism. In particular, the fixing bolt can be guided out of the recess by actuating the rocker mechanism. Preferably, the rocker mechanism comprises a rocker or a pull lever, which is designed as a two-sided lever. The fixing bolt is preferably attached to the rocker. In particular, the rocker is rotatably mounted on a fulcrum on the pressure chamber or the vacuum tank. In particular, the rocker mechanism comprises a spring unit which preloads the fixing bolt, in particular into a closed position, so that the rocker mechanism is automatically preloaded into an arrested position. Preferably, the spring unit comprises a coil spring or the like. Advantageously, through that, the rocker mechanism can be operated with one hand. In particular, the rocker mechanism comprises an attachment flange by which it is held to the pressure chamber. Preferably, the attachment flange has an opening through which the fixing bolt can be passed.

In particular, the device comprises a controller which is designed to control the pump. In particular, the controller enables an alternating pressure program to be carried out, which is designed in particular as a massage program, and in particular further functions, such as changing the inclination of the second lying surface or moving the pressure chamber. Advantageously, the controller comprises an operating unit, which is designed in particular as a tablet. Preferably, the operating means can be connected wirelessly, e.g., by radio connection via W-LAN, Bluetooth or similar.

Preferably, the controller comprises a measuring unit with at least one pressure sensor for monitoring the pressure in the pressure chamber. The pressure sensor is advantageously designed as a pressure transmitter. In particular, the measuring unit can include other sensors, in particular for recording user data such as height, girth, weight or the like. Advantageously, the data that can be recorded by the measuring unit can be taken into account in the application in order to adapt and adjust the application to the user. Beyond that, the position of the pressure vessel can be monitored through the measuring unit during use. The position can be monitored technically by limit switches and/or overrun switches.

Preferably, the device comprises a second actuator which is designed to move the pressure chamber, designed as a pressure vessel, parallel to the lying surface from the first state to the second state and vice versa. In particular, the controller is designed to control the second actuator. In one embodiment, the pressure vessel can also be moved manually.

In particular, the controller is designed to create a control program for the pump. Advantageously, the controller is designed to control the pump according to the control program, and wherein the controller is designed to use multiple user parameters when creating the control program. In particular, the user parameters comprise values such as age, gender, weight, height, lifestyle habits such as being a smoker, exercise and sport activity. Expediently, the controller is designed to use measurement results from the measuring unit when creating the control program. Therein, the user data can be recorded through the measuring unit or through manual input. Preferably, the device comprises a base frame. In particular, the base frame can be divided into two parts. Advantageously, the parts can be transported individually so that the parts of the base frame can also be transported individually. Advantageously, convenient transportation is enabled through that.

According to one disclosed embodiment, the controller can be designed to create a control program for the pump. Several user parameters can be used during creation. The user parameters can comprise, for example, a user's age, gender, weight, height and lifestyle habits. For example, the user parameters can be previously gathered from the user. The controller provided in one embodiment can preferably comprise a tablet computer or be designed as such. The controller is preferably designed to communicate wirelessly, preferably via WLAN, with other electronic components.

Besides that, measurement results can also be used when creating the control program. The measurement results can, for example, result from measurements taken by the user. This means that the control program can be adapted both to a specific user and to the current status of the user.

Further features and advantages of the disclosed embodiments are described below with reference to the drawings. Therein, the same reference symbols are used for identical or similar parts and for parts with identical or similar functions.

### BRIEF DESCRIPTION OF THE DRAWINGS

FIG. 1 is a schematic perspective view of a device according to one embodiment;

FIG. 2 is a schematic perspective view of the device in FIG. 1 with the pressure vessel pulled out parallel to the lying surface in the first state;

FIG. 3 is a schematic perspective detail view of a part of the device in FIG. 1;

FIG. 4 is a schematic perspective view of a device according to one embodiment;

FIG. 5 is a schematic perspective view of the device in FIG. 4 with the pressure vessel pulled out parallel to the lying surface;

FIG. 6 is a schematic perspective view of the device in FIG. 4 with the pressure vessel pushed inside;

FIG. 7 is a schematic sectional view of the rocker mechanism for actuating the fixing bolt;

FIG. 8 is a schematic sectional view of the rocker mechanism for actuating the fixing bolt;

FIG. 9 is a schematic sectional view of the rocker mechanism for actuating the fixing bolt;

FIG. 10 is a purely schematic view of the fastener with the contact surface and with the contact length of the fabric mechanism; and

FIG. 11 is a further perspective view to visualize the contact surface with the contact length of the fabric mechanism.

### DETAILED DESCRIPTION

It is not necessary that a device according to the invention have all the features described below. It is also possible for a device according to the invention to have only individual features of the example embodiments described below.

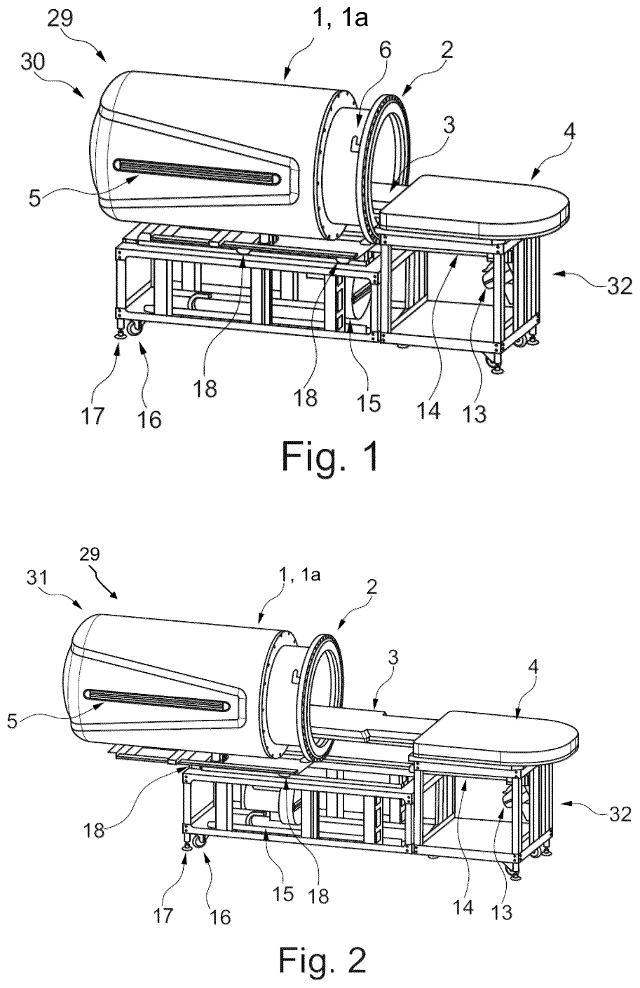

FIG. 1 is a schematic perspective view of a device 29 according to one example embodiment. The device 29 comprises a pressure chamber 1 designed as a pressure vessel 1a, a pump 15 and a lying surface 3, 4. The lying surface 3, 4 comprises a first part 3 and a second part 4. The first part 3 of the lying surface 3, 4 is arranged in the pressure chamber 1 and the second part 4 of the lying surface 3, 4 is arranged outside the pressure chamber 1. Further, the device 29 has a fastener 2, which is arranged at the entrance opening of the pressure chamber 1. The fastener 2 can be moved from an open to a closed state and vice versa in order to seal a human body arranged on the lying surface 3, 4 in an airtight manner with respect to an environment, wherein the pump 15 is designed to pump a gas or gas mixture, such as ambient air, into the pressure chamber 1 and thus increase a pressure within the pressure chamber 1 and also to pump it out of the pressure chamber 1 and thus reduce a pressure within the pressure chamber 1. Advantageously, through the changing of pressure, a massage effect can be created, which has a particularly positive effect on the appearance of the skin.

The device 29 further comprises a base frame 32 on which the lying surface 3, 4 and the pressure chamber 1 are arranged. Here, the pressure chamber 1 can be moved relative to the lying surface 3, 4 and the base frame 32 via a rail structure 18. Through that, a user can first position themselves comfortably on the lying surface 3, 4 and lie down and then the pressure chamber 1 can be moved from the first or retracted state 30 shown in FIG. 1 to the extended state 31 shown in FIG. 2. Herein, the movement is realized either manually or via a second actuator 14, which is designed here as an electric drive and can be controlled via a controller 7 (FIG. 4). In the first state 30, the first lying surface 3 is arranged completely inside the pressure chamber 1 and in the second extended state 31, less than half of its length is arranged outside the pressure chamber 1.

Here, it is particularly advantageous that the position of the lying surface 3, 4 in relation to the user does not change. Through this, the device 29 is prevented from tipping over.

LED strips 5, 12 are attached to the pressure vessel 1 and to the base frame 32. These can be used, for example, to display an operating state of the device 29 and can be activated for this purpose by the controller 7.

Here, the inclination of the second part 4 of the lying surface 3, 4 can be adjusted relative to the first part 4 of the lying surface 3, 4. Further, the height of the lying surface 3, 4 can be adjusted here to increase comfort for the user. At the same time, the position of the human body in relation to the fastener 2 can be optimized here so that the fastener 2 seals against the torso. The two lying surfaces 3 and 4 are in permanent or constant contact, so that there is no gap between them. The lying surface 3, 4 can be adjusted via a first actuator 13. Here, the first actuator 13 is designed as an electric drive, which is arranged on the base 32 and can be controlled via the controller 7.

Caster wheels 16 are arranged on the base frame 32 to enable easy and convenient transportation. The caster wheels 16 can be lowered onto adjustable feet 17 to enable firm standing of the device 29.

FIG. 2 shows a schematic perspective view of the device 29 in FIG. 1 with the pressure vessel 1a pulled out parallel to the lying surface 3, 4 or in the extended state 31.

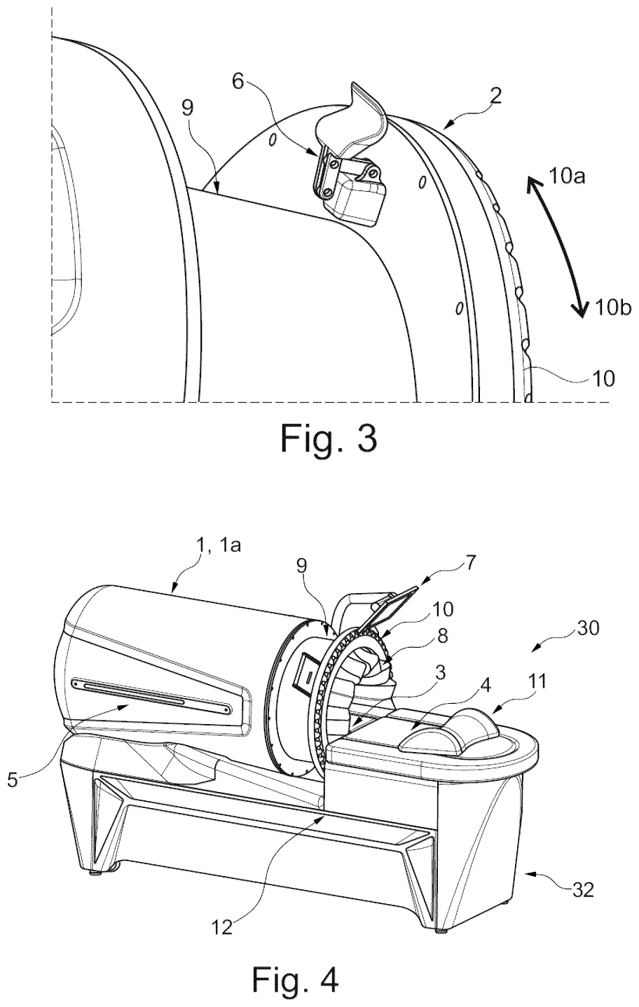

FIG. 3 a schematic perspective detail view of a part of the device 29 in FIG. 1. Here, the fastener 2 comprises a rocker mechanism 6 by which the position of the fastening ring 10 can be arrested. The fastener 2 is arranged here on a holding rim 9. The fastening ring 10 can rotate in both directions 10a, 10b and can be moved from an open to a closed state.

FIG. 4 shows a schematic perspective view of a device 29 according to a further embodiment of the invention. Here, the device 29 comprises a controller 7, which comprises a tablet 7 for controlling the device 29. Here, the tablet 7 is arranged on an arm so that the user of the device 29 can control it from the lying surface 3, 4.

Here, the airtight fabric mechanism 8 of the fastener 2 is also shown. When moving the fastener to the closed state, the tube mechanism 8 is twisted. The fabric mechanism 8 wraps around the human torso and seals the pressure chamber 1 from the environment. Due to the design, closing can be achieved here by rotating the fastening ring 10 in both directions 10a, 10b.

Here, a neck pillow 11 on the lying surface 3, 4 for supporting the user's neck and head is also shown. Here, the device 29 is shown in the retracted state 30.

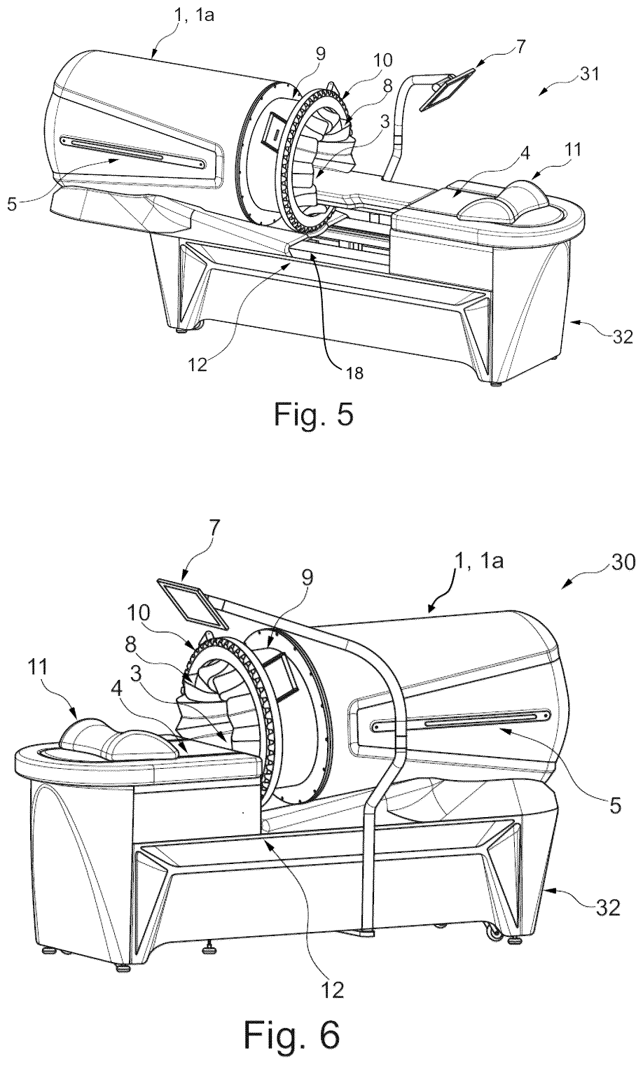

FIG. 5 and FIG. 6 show further schematic perspective views of the device 29 of FIG. 4 in the retracted state 30 and extended state 31. Here, the arrangement of the controller 7 within the user's reach is clearly recognizable.

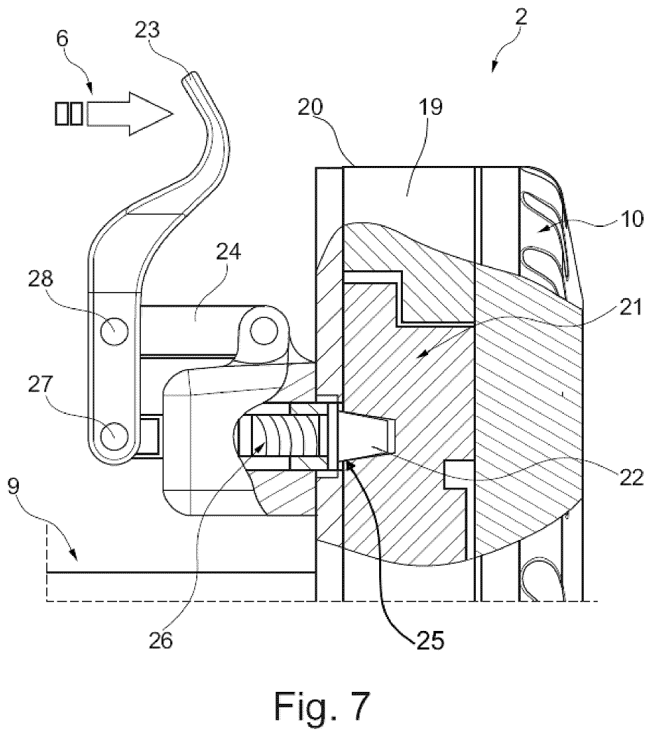

FIG. 7 shows a schematic sectional view of the rocker mechanism 6 of the fastener 2 for actuating a fixing bolt 22. Here, the fastener 2 is held on the holding rim 9 on the pressure chamber 1. Here, the fastener 2 comprises the assembly with the fastening ring 10 and the assembly with the rocker mechanism 6. The assembly with the fastening ring 10 also comprises a flange 20, on which the assembly is held on the holding rim 9, and a clamping ring 19. The fastening ring 10 comprises a grid ring 21 in which a plurality of recesses 25 are arranged on the axial front surface distributed over the circumference, wherein one recess 25 is shown here schematically in sectional view.

Here, the rocker mechanism 6 comprises the fixing bolt 22. The fixing bolt 22 can engage in the recess 25 and thereby lock the fastening ring 10 against rotation, so that the fabric mechanism 8, which is not shown here, is fixed in a twisted position on the user's body in such a way that there is an airtight connection which no longer loosens.

The fixing bolt 22 is held on a lever joint 27, which further connects a movement of the fixing bolt 22 via a compensating joint 28, a deflection rail 24 and a pull lever 23. Through pulling on the pull lever 23, the fixing bolt 22 is lifted out of the recess 25.

The fixing bolt 22 is preloaded through a spring 26 of a spring unit 26, which is designed here as a coil spring 26, which preloads the fixing bolt 22 into the locked position so that it is automatically pushed back into the locked position when the pull lever 23 is released. The fixing lever is enclosed by the attachment flange.

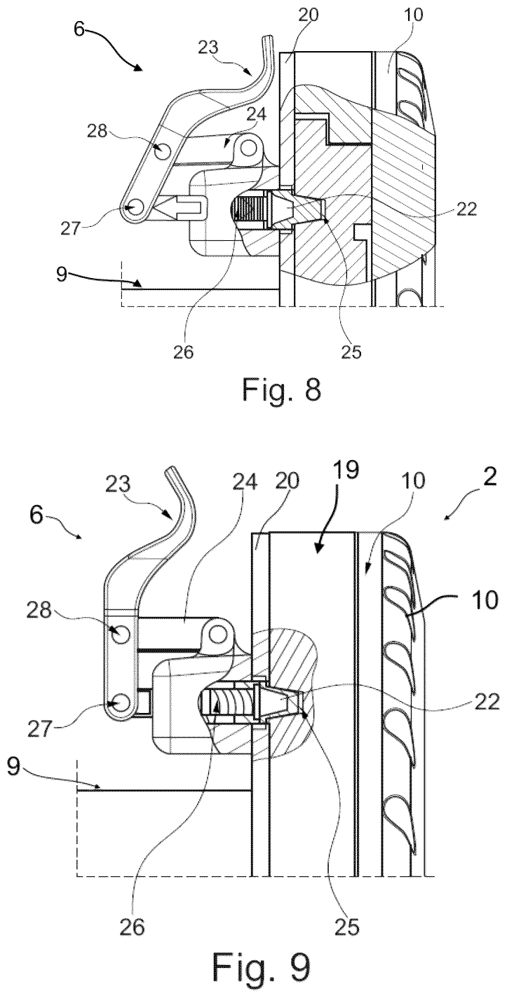

FIG. 8 shows a further schematic sectional view of the rocker mechanism 6 for operating the fixing bolt 22 in the operated position, and FIG. 9 shows another schematic sectional view of the rocker mechanism 6 for operating the fixing bolt 22 in the non-operated position.

In particular, it is possible that the maximum negative pressure in the pressure chamber 1 is up to −130 mbar. The maximum overpressure can be +50 mbar or also up to +130 mbar, for example. Therein, the pressure is always specified relative to the ambient pressure. The material of the device 29 can be stainless steel, aluminum and carbon. The base frame 32 of the device 29 can be a load-bearing stainless steel frame with aluminum base plates for control elements and in particular the controller 7 and drive peripherals, in particular the actuators 13, 14. The base frame 32 is movable, for example with four steerable caster wheels 16, and can be divided into two separate parts for local circumstances which are unusual for the purpose. The base frame 32 is then fixed in its place, in particular by feet 17, which can be adjusted by unscrewing. The caster wheels 16 then no longer have any function. The second part of the lying surface 3, 4 of the upper body can be moved upwards for comfortable sitting by means of the first actuator 13. The backrest adjustment is carried out via the control unit 7.

The pressure chamber 1, designed as pressure vessel 1a, can also be extended and retracted via a second actuator 14. The pressure vessel 1a, made of carbon, runs in particular on a rail structure 18 on which the pump 15, designed here as a side channel compressor, also travels. The controller 7 comprises at least one main control unit, a PLC control unit with a RCCB. The various pressures are generated in particular by means of the pump 15, which is designed as a side channel compressor, in conjunction with the frequency converter and are preferably constantly monitored.

During use, the position of the pressure tank 1a as well as the actual pressure values and backrest position can be constantly monitored. Technically, the (movement) position of the pressure vessel 1a is preferably monitored by limit switches.

During the process, the user is preferably protected through overload switches. During use, the pressure can be monitored with a pressure transmitter.

The application of alternating pressure massage in particular serves primarily to improve the appearance of the skin. A good side effect of this massage can be the stimulation of the lymph flow and the positive effect on the connective tissue of the lower body. The pressure vessel 1a is sealed in particular by an airtight fabric, especially a fabric 8, for example similar to the airbag fabric known from the automotive industry.

The core of the controller 7, in particular the control system, is expediently the software based on real measurements and/or user parameters gathered from the user, which designs the most advantageous and, where appropriate, best possible application for each user at the time of the measurement. Preferably, a basic program based on customer-specific parameters such as age, gender, weight, height and lifestyle habits is stored in a memory of the control unit. This basic program can then be replaced or refined with daily measurements if necessary. This means that each individual application can be unique based on the user's basic program and daily status, i.e., preferably individually adapted to the user's appearance current for the day.

In particular, the device 29 is operated by the user or operator or machine operator via a tablet PC 7. In the basic position, the pressure vessel 1a is preferably extended and the second lying surface 4 is in particular raised. The user preferably sits on the narrow part of the first lying surface 3 and leans back. On the tablet PC 7, they now start the movement or retraction of the pressure tank 1a in particular. When the target position or end position is reached, the software can switch over. In particular, the user operates the rocker mechanism 6 or the arresting rocker of the fastener 2 or the fastening ring 10 and closes the fastening ring 10 at the height of the navel with the airtight fabric 8 or material 8. If desired, they can lower the second lying surface and in particular the backrest slightly. Through the releasing of the pull lever 23 or the rocker, the fastening ring is preferably arrested. A preferably installed program can now be started on the surface of the tablet PC 7, which can run fully automatically. During the application, the user can in particular have his treatment progress be displayed, preferably follow an on-screen animation and/or listen to “relaxed music” or relaxation music.

Preferably, the fastener 2, in particular the fastening mechanism, can be fastened not only in one direction 10a, but in particular in both directions 10a, 10b. Constructively, this is solved by a rocker mechanism 6, whose spring-loaded fixing bolt 22 or locking bolt 26 slides through the fastening flange into an extra adjustment grid with a large number of recesses 25 located in the fastening ring 10. Through operating of the pull lever 23 or the rocker, the fixing bolt 22 is released and the fastening ring 10 can be moved freely and very smoothly in both (fastening/opening) directions 10a, 10b. Aluminum is preferably used as the material for the fastening ring 10. The smooth-running capability is preferably achieved by a large number of internally installed ball bearings.

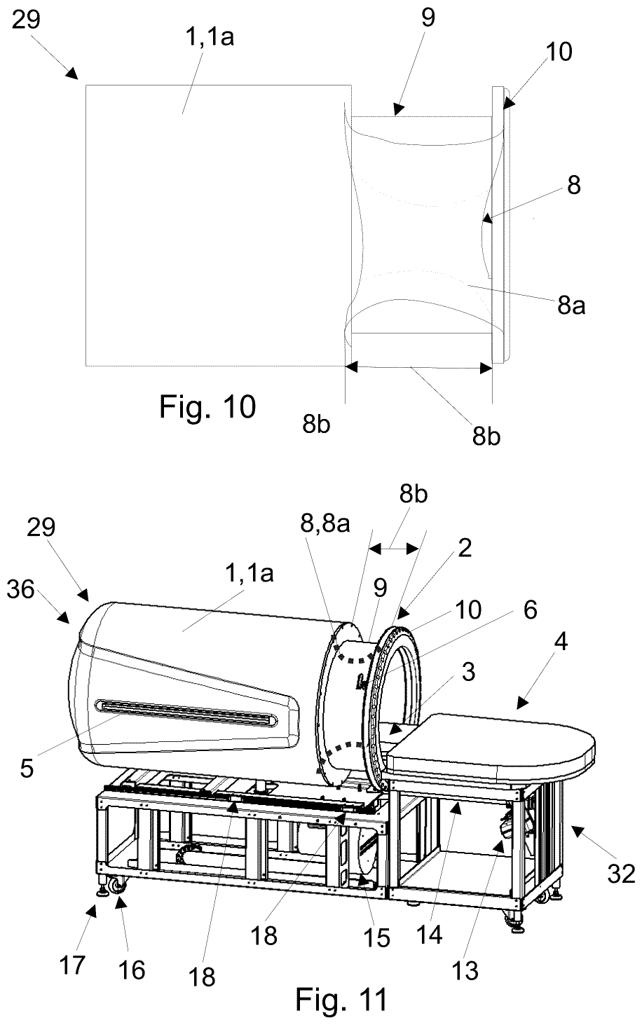

FIG. 10 shows a purely schematic view of the fastener 2 with a contact surface 8a of the airtight fabric mechanism 8, which rests against the user's torso over a contact length 8b. Here, the support length 8b is approximately 8 cm. This allows a particularly high level of impermeability of the fastener 2 on the user's torso in relation to the environment to be achieved. Further, through the long support length 8b it is achieved that the pressure on the user's torso is low. Through that, a negative pressure of up to −130 mbar can be generated by the device 29 during operation.

FIG. 11 shows a further perspective view for visualizing the contact surface 8a with the contact length 8b of the airtight fabric mechanism 8. Here it can be seen that the contact surface 8a extends over the entire length of the rim 9.

What is claimed is:

1. A device, comprising:
a pressure chamber including a fastener movable between an open state and a closed state;
a pump to pump gas into the pressure chamber to increase pressure within the pressure chamber and to pump gas out of the pressure chamber to reduce pressure within the pressure chamber; and
a lying surface including a first part arrangeable in the pressure chamber and a second part arranged outside the pressure chamber, wherein the fastener, in the closed state, is configured to close the pressure chamber around a human body arranged on the lying surface in an airtight manner in relation to an environment.

2. The device of claim 1, wherein the pressure chamber comprises a pressure vessel moveable between a first state in which more than three quarters of a length of the first part of the lying surface is arranged inside the pressure chamber, and a second state in which less than half of the length of the first part of the lying surface is arranged outside the pressure chamber.

3. The device of claim 2, wherein, in the first state, an entirety of the first part of the lying surface is arranged inside the pressure chamber.

4. The device of claim 2, wherein, in the second state, an entirety of the first part of the lying surface is arranged outside the pressure chamber.

5. The device of claim 2, further comprising a rail structure and a base frame, the pressure vessel being movable over the rail structure relative to the base frame, the first and second parts of the lying surface being fixedly attached to the base frame.

6. The device of claim 1, wherein an inclination of the second part of the lying surface relative to the first part of the lying surface is adjustable.

7. The device of claim 1, wherein the first and second parts of the lying surface are height-adjustable.

8. The device of claim 1, wherein the first and second parts of the lying surface are touching each other.

9. The device of claim 1, wherein the fastener comprises a fastening ring and the fastener is movable from the open state to the closed state by a rotation of the fastening ring in either of two directions of rotation.

10. The device of claim 9, wherein the fastening ring includes recesses its circumference and the fastener comprises a fixing bolt insertable into one of the recesses to prevent rotation of the fastening ring.

11. The device of claim 10, wherein the fastener further comprises a rocker mechanism and the fixing bolt is removable from the one of the recesses by an actuation of the rocker mechanism.

12. The device of claim 1, further comprising:
a controller to control the pump.

13. The device of claim 12, wherein the controller comprises a measuring unit with at least one pressure sensor to monitor the pressure in the pressure chamber.

14. The device of claim 13, wherein the controller is configured to create for the pump a control program based on user parameters and to control the pump according to the control program.

15. The device of claim 14, wherein the controller is configured to create the control program based on measurement results.

16. The device of claim 12, wherein the pressure chamber comprises a pressure vessel moveable between a first state in which more than three quarters of a length of the first part of the lying surface is arranged inside the pressure chamber, and a second state in which less than half of the length of the first part of the lying surface is arranged outside the pressure chamber,
the device further comprising an actuator to move the pressure chamber parallel to the lying surface between the first and second states, wherein the controller is configured to control the actuator.

17. The device of claim 16, wherein the controller is configured to create for the pump a control program based on user parameters and to control the pump according to the control program.

18. The device of claim 17, wherein the controller is configured to create the control program based on measurement results.

19. The device of claim 1, further comprising a base frame including first and second parts.
